(19) United States
(12) Patent Application Publication
(10) Pub. No.: US 2024/0368210 A1
(43) Pub. Date: Nov. 7, 2024

(54) PROCESSES FOR THE PREPARATION OF ESTETROL AND INTERMEDIATES THEREOF

(71) Applicant: NEWCHEM S.P.A., Milano (IT)

(72) Inventors: Francesco Fabris, Milano (IT); Francesco Farinella, Milano (IT); Luca Gambarin, Milano (IT); Massimo Merlo, Milano (IT); Gianfranco Lopopolo, Milano (IT); Emanuele Attolino, Milano (IT)

(73) Assignee: NEWCHEM S.P.A., Milano (IT)

(21) Appl. No.: 18/580,851

(22) PCT Filed: Jul. 20, 2022
(86) PCT No.: PCT/EP2022/070292

§ 371 Date: Jan. 19, 2024

(30) Foreign Application Priority Data
Jul. 23, 2021 (IT) 102021000019631

(51) Int. Cl. C07J 1/00

(52) U.S. Cl. CPC C07J 1/007

(57) ABSTRACT

A process for the preparation of a compound of formula (IIa) is described where A is a silyl protecting group, in high diastereoisomeric purity and in crystalline form, comprising the crystallization of a mixture of the compound of formula (IIa) and its isomer of formula (IIb), where A is a silyl protecting group, from an ethereal solvent. The invention also provides a process for preparing Estetrol of formula (I) in high diastereoisomeric purity, comprising the use of the compound of formula (IIa), in a crystalline form, as a starting material.

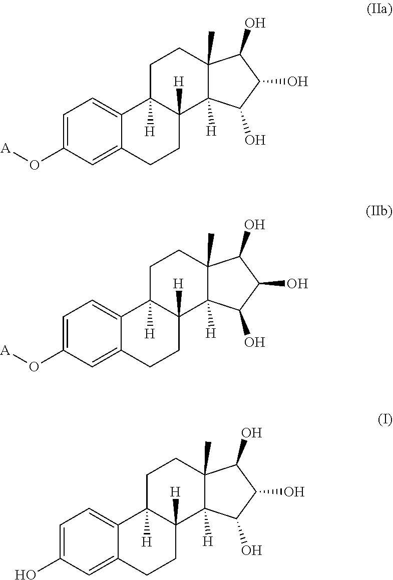

### FIELD OF THE INVENTION

The present invention relates to a process for the preparation of an isomerically pure crystalline intermediate useful to produce Estetrol.

### BACKGROUND OF THE INVENTION

Estetrol [estra-1,3,5 (10)-trien-3, 15α, 16α, 17β-tetraol] is a steroidal hormone with weak estrogenic activity that is endogenously produced by the fetal liver in detectable levels only during pregnancy in women. It has the following structural formula (I).

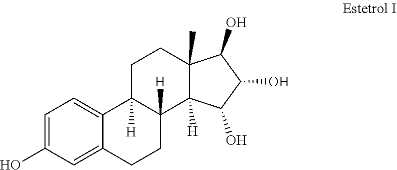

Estetrol of formula (I) has been recently approved by the FDA as a contraceptive drug in combination with Drospirenone and is still in the clinical phase also for the treatment of menopause.

The synthesis of Estetrol of formula (I), is known both in the scientific and patent literature. Several synthetic routes for Estetrol published over the years will be described below.

Fishman et al. J. Org. Chem. 1968, 33, 3133-3135 published a synthesis of Estetrol of formula (I), on a laboratory scale, starting from Δ-15-Estrone of formula (IV), according to the following Scheme 1:

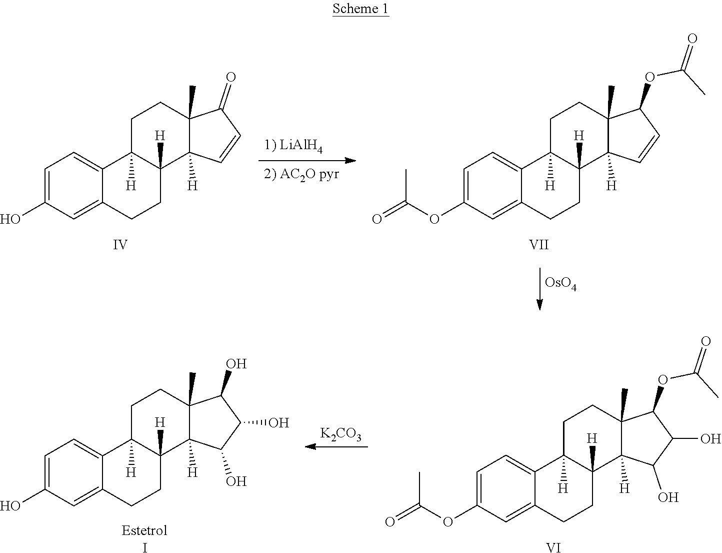

Fishman's synthesis first of all involves the reduction of the carbonyl in C17 of the Δ-15-Estrone of formula (IV), with LiAlH₄, followed by the protection of the hydroxyl groups in position 3 and 17 as acetate esters to provide the protected Δ-15-Estradiol of formula (VII). The cis-dihydroxylation of the double bond in position C15-C16 of the protected Δ-15-Estradiol of formula (VII) with osmium tetroxide gives a crude containing the protected Estetrol of formula (VI). However, the paper does not provide any details about the selectivity of the cis-dihydroxylation, which can obviously occur both above and below the planar C═C double bond.

Finally, the protecting groups are cleaved to obtain Estetrol of formula (I). The total yield of the process is 7%.

Subsequently Nambara et al., Steroids 1976, 27, 111-121 disclosed a synthesis of Estetrol of formula (I), focused on the formation of the C═C double bond in the C15-C16 positions, by bromination/debromination reaction starting from Estrone of formula (V) with the route of synthesis reported below in Scheme 2.

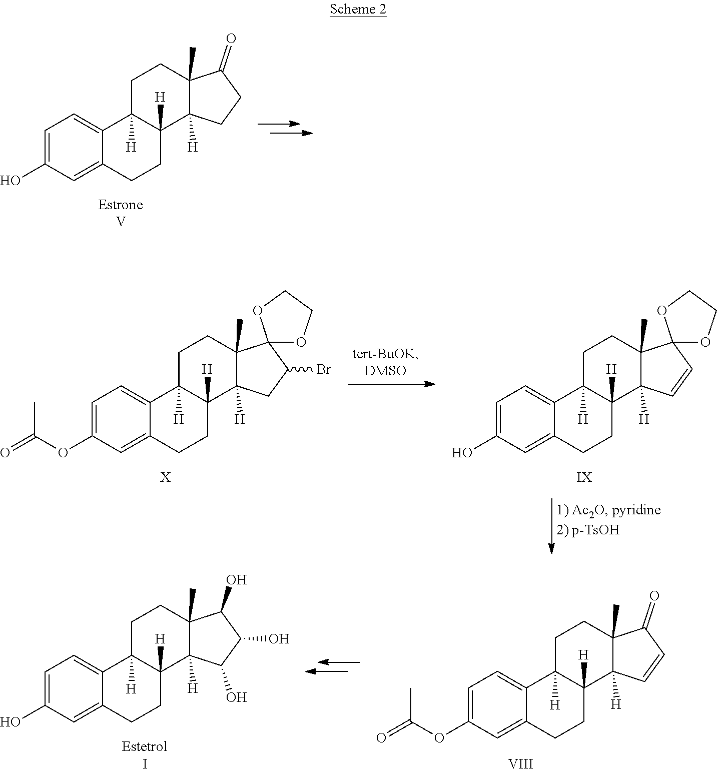

In this synthesis, the Estrone of formula (V) is first protected to the carbonyl in C17 with ethylene glycol and subsequently at C3 by acetylation, then the protected intermediate is subjected to bromination in position C16 to give the brominated compound of formula (X). The elimination with potassium terbutoxide in DMSO yields to the formation of the compound of formula (IX) having the double bond in the C15-C16 positions.

The deprotection in C17 and the protection in C3 of the compound of formula (IX), yields to the formation of the protected Δ-15 Estrone of formula (VIII).

Finally, the synthesis continues with the same process described by Fishman to give Estetrol of formula (I). Unfortunately, neither the total yield of the process nor details on selectivity of the cis-dihydroxylation reaction are provided in this study.

Several years later, Suzuki et al., Steroids, 1995, 60. 277-284 shed light on the selectivity of the cis-dihydroxylation reaction of the C15-C16 double bond of protected Δ-15 Estradiol, of formula (VII) according to the same process described by Nambara et al., (Scheme 3). In the paper has been described how, after having isolated the end of the reaction crude, the oily residue can be purified first by chromatography and then by crystallization from chloroform/ether which gives the two isomers 15α, 16α of formula (VIa) and 15β, 16β of formula (VIb) in the ratio of 74:26.

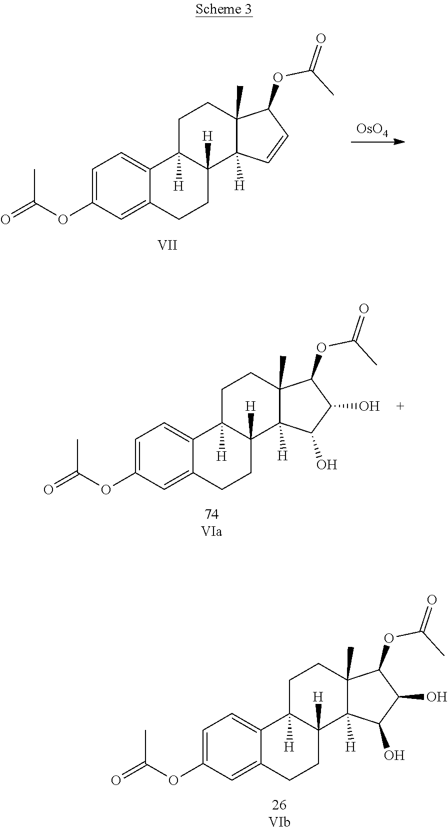

In WO2004/041839 a multistep process has been subsequently disclosed for the preparation of the intermediate of formula (XII) which by after cis-dihydroxylation reaction under Nambara conditions provides the mixture of stereoisomers of the compounds of formula (XI) (Scheme 4).

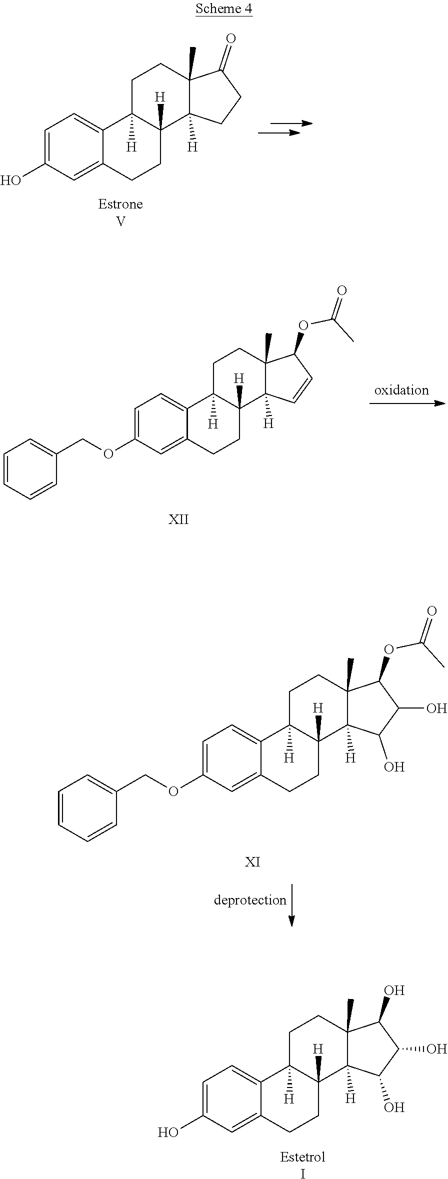

Specifically, WO2004/041839 gives a yield of the cis dihydroxylation reaction of 43% after three crystallizations from heptane/ethyl acetate/ethanol (2:1:1), necessary to purify the product from the 15β, 16β isomer of formula (XIb).

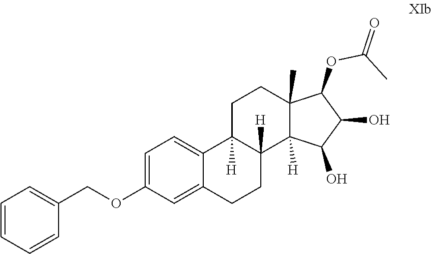

WO2013/012328 discloses a process for the preparation of Estetrol of formula (I) with the process reported in Scheme 5.

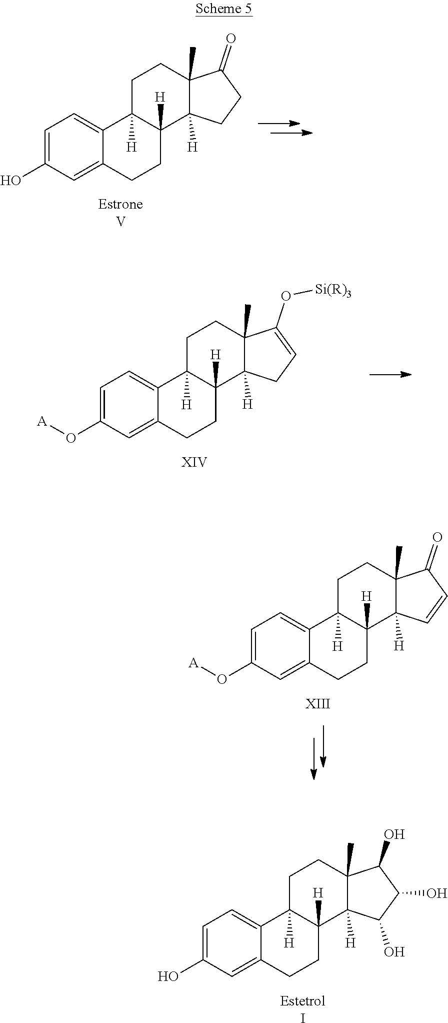

The process involves the conversion of Estrone of formula (V) into 4-15 Estrone with the OH in C3 protected, of formula (XIII) in three steps instead of the five steps known in prior art (see WO2004/041839A2 for instance), through formation of the silyl enol ether intermediate of formula (XIV) and then its oxidation with IBX, IBS, iodine or oxygen in presence of a palladium catalyst, in particular Pd(OAc)₂. The synthesis continues with the process already described in WO2004/041839 to give Estetrol of formula (I).

This process highly shortens the synthesis process, but makes use of large quantities of palladium, with the following use of laborious operations to recover the precious metal and considerable increase in the cost of the process which resulted to be not suitable for synthesis on an industrial scale.

WO2013/050553 discloses a process for the preparation of Estetrol of formula (I) with traces or no presence of its 15β, 16β isomer of formula (Ib) starting from a protected Δ-15 Estrone of formula (XV), where A is a silyl protecting group (Scheme 6),

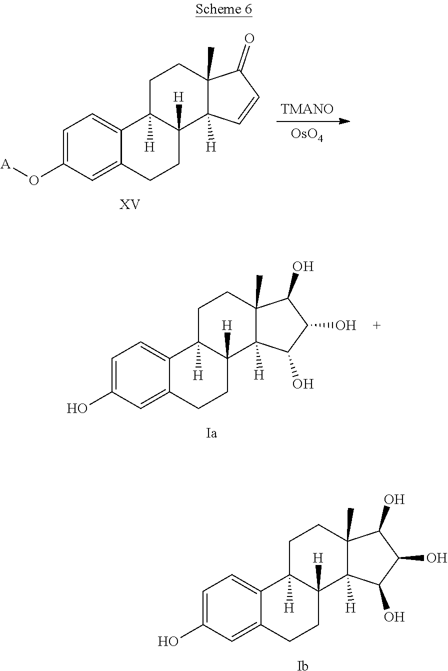

by cis-dihydroxylation reaction using KMnO₄, OsO₄, H₂O₂ or I₂/Ag(OAc)₂ as oxidants, followed by the deprotection at C3.

However, neither data nor examples to support this process have been reported.

WO2013/034780 discloses a process for the preparation of Estetrol of formula (I) with excellent selectivity performing the cis dihydroxylation reaction of the intermediate of formula (XVI) wherein the hydroxyl group in position C17 is not protected. The process avoids the need to use protecting groups at the C17 position avoiding the protection/deprotection reactions and providing a 15α, 16α: 15β, 16β selectivity equal to 90:10. (Scheme 7).

Despite the good result of selectivity, the inventors do not provide details neither on the isolation nor characterize the 15α, 16α isomer of formula (IIa).

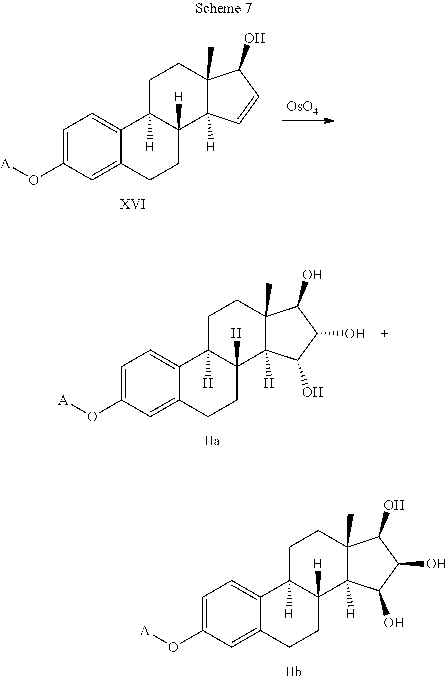

WO2015/040051 discloses a process to produce Estetrol of formula (I) by cis-dihydroxylation reaction of the double bond in C15-C16 of the compound of formula (XVIII) with very good selectivity results. The reason is attributed to the nature of the R² protective group in position C17. However, the synthetic steps related to the insertion and cleavage of the protective group are often challenging, and specifically require high temperatures and long reaction times (Scheme 8).

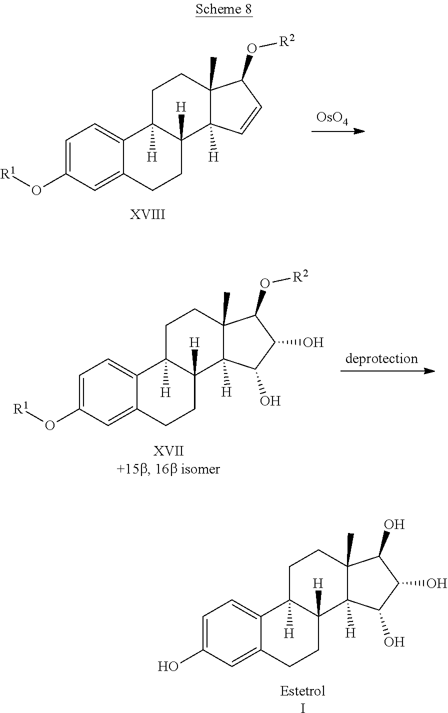

Despite the enormous work reported in the literature on the synthesis of Estetrol of formula (I), still exists the need to have a sustainable process for the preparation in pure form of the 15α, 16α isomer reducing the presence of the 15β, 16β isomer without the need of processes involving several, long and expensive reactions to eliminate the protective groups and purifications.

### DESCRIPTION OF FIGURES

FIG. 1: XRPD profile of the compound of formula (II), in crystalline form, here defined Form I, where the most intense peaks, at about 2θ°, are: 6.8±0.2°, 10.2±0.2°, 13.6±0.2°, 14.6±0.2°, 15.8±0.2°, measured at a wavelength of 1.5418 Å.

FIG. 2: DSC thermogram of the compound of formula (II), in crystalline form, here defined as Form 1. The endothermic peak at about 225° C. indicates the melting process.

### DESCRIPTION OF THE INVENTION

A first object of the present invention is a process for the preparation of a compound of formula (IIa),

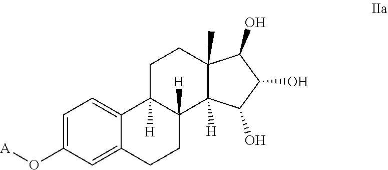

wherein A is a silyl protecting group, in high diastereoisomeric purity and in crystalline form, comprising the crystallization of a mixture of the compound of formula (IIa) and its isomer of formula (IIb),

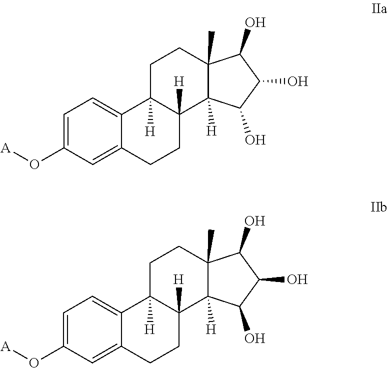

where A is a silyl protecting group, from an ethereal solvent comprising at least one linear or branched alkyl (C1-C5) ether and at least one cyclic (C4-C6)cycloaliphatic ether. The use of silyls as protecting group of the hydroxyl group is known by the person skilled in the art and described by Green and Wuts in “Protective Groups in Organic Synthesis”, 4th Edition (2007), Ed. John Wiley & Sons (ISBN 0-471-69754-0).

Specifically, the hydroxyl protecting group A of the present invention is a silyl group of formula-Si(R)₃, wherein R is independently selected from the group comprising (C1-C4)alkyl, (C6-C10)aryl, (C1-C4)alkyl-(C6-C10)aryl and (C6-C10)aryl-(C1-C4)alkyl. Preferably, the silyl groups of formula-Si(R)₃ are selected from the group comprising trimethylsilyl, triethylsilyl, tert-butyldimethylsilyl, tri-isopropylsilyl, diphenyltertbutylsilyl, diphenylmethylsilyl and phenyldimethylsilyl.

Examples of a linear or branched (C1-C5)alkyl ether are diethyl ether, diisopropyl ether and methyl tert-butyl ether or a mixture thereof. Preferably the linear or branched (C1-C5) alkyl ether solvent is diisopropyl ether.

Examples of (C4-C6)cycloaliphatic ethereal solvents are tetrahydrofuran and 1,4-dioxane or a mixture thereof. Preferably the cycloaliphatic ethereal (C4-C6) solvent is tetrahydrofuran.

In the process of the invention, the cycloaliphatic (C4-C6) ether and the linear or branched (C1-C5)alkyl ether are, respectively, solvent and antisolvent.

In a preferred aspect, the process object of the present invention, can be performed by dissolving a mixture of the compound of formula (IIa) and its isomer of formula (IIb), in a molar ratio between (IIa) and (IIb) comprised between 85/15 and 95/5, preferably around 90/10. in an (C4-C6)cycloaliphatic ethereal solvent in an amount comprised between 1 to 10 mL per gram of mixture preferably comprised between 1 to 5 mL per gram of mixture.

The dissolution can be performed at a temperature comprised between 20° C. to 55° C., preferably under inert atmosphere, then the mixture is preferably kept under stirring at a temperature comprised between 20° C. up to the reflux temperature of the solution.

Once the solution is obtained, the mixture is cooled under stirring to a temperature comprised between 25° C. to 0° C. Successively an amount of antisolvent is added comprised between 1 to 20 mL per gram of mixture of starting material (IIa) and (IIb), preferably comprised between 1 and 10 mL of antisolvent per gram of the starting mixture of compound (IIa) and (IIb).

The ratio between the solvent and the antisolvent of the process of the invention, given in v/v, may range from 1:1 to 1:10, preferably from 1:3 to 1:7.

Preferably the ethereal solvent of the present invention in tetrahydrofuran. Preferably the ether antisolvent of the present invention is diisopropyl ether.

The crystalline solid of formula (IIa), obtained by the process of the present invention, can be recovered by optional addition of a further aliquot of ethereal antisolvent, as defined above, in order to fluidize the dispersion.

The crystalline solid of formula (IIa), obtained by the process of the present invention, can be recovered following known techniques, such as filtration or centrifugation.

The crystalline solid of formula (IIa), wet or dry, obtained by the process of the present invention can optionally be subjected again to the process object of the present invention.

The crystalline solid of formula (IIa), obtained by the process of the present invention, can be dried at a temperature ranging from 25° C. to 80° C., following known processes, preferably from 35° C. to 65° C., optionally under vacuum. The crystalline solid of formula (IIa), obtained by the process of the present invention, is characterized by an impurity content of formula (IIb) in an amount lower than 0.5% (area % HPLC), preferably lower than 0.2% (area %), determined by HPLC analysis, so that it can be considered of high diastereomeric purity.

Therefore, a further aspect of the present invention is a compound of formula (IIa) where A is defined above, characterized in that (i) it is in crystalline form, (ii) it has a diastereomeric purity determined by HPLC analysis of at least 99.5% (area %), and (iii) it contains the isomer 15β, 16β of formula (IIb), where A is defined above, in an amount lower than 0.5% (area %), preferably lower than 0.2% (area %).

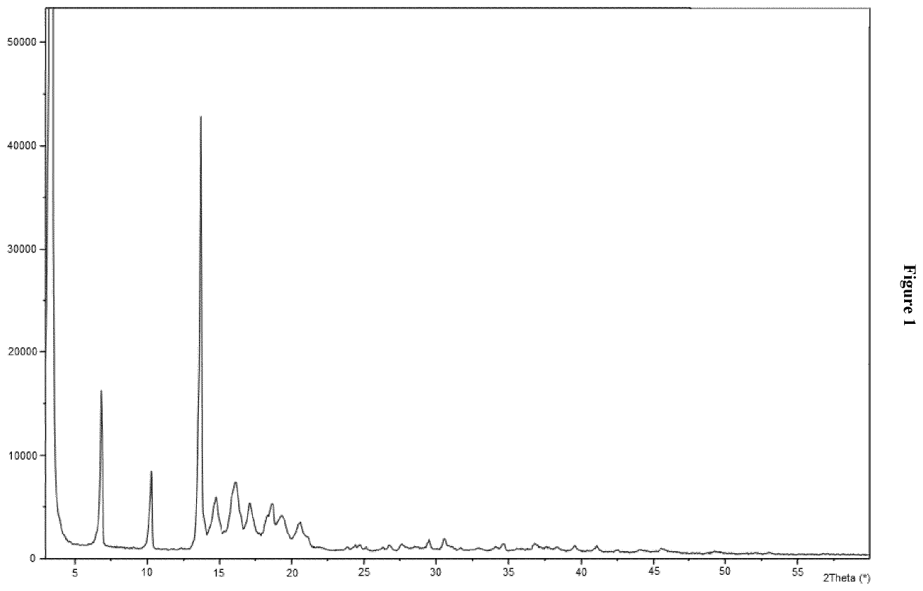

In particular, a further object of the present invention is a compound of formula (IIa) as defined above having formula (II)

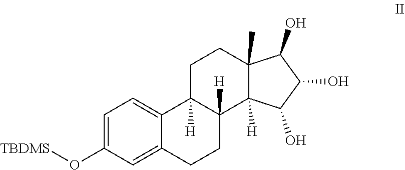

where TBDMS represents the tert-butyldimethylsilyl group, where the crystalline form, herein defined Form I, has an XRPD profile as shown in FIG. 1, where the most intense peaks, at about 2θ°, are: 6.8±0.2°, 10.2±0.2°, 13.6±0.2°, 14.6±0.2°, 15.8±0.2°, measured at a wavelength of 1.5418 Å.

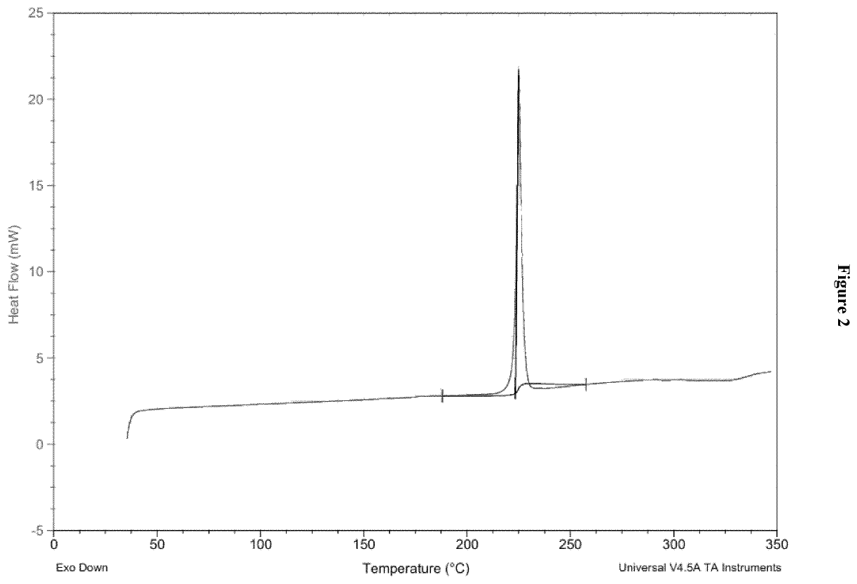

The crystalline form of the compound of formula (II), herein defined Form I, presents a DSC thermogram as shown in FIG. 2, where the endothermic peak at about 225° C. indicates the melting process.

The crystalline form of a compound of formula (II), herein defined Form I, is characterized by a water content lower than 0.5% by weight preferably lower than 0.2% by weight, so that it can be defined essentially anhydrous.

The compound of formula (II), in crystalline Form I, according to the present invention, has a diastereoisomeric purity determined by HPLC, greater than or equal to 99.5% (area %).

In particular, the compound of formula (II), in crystalline Form I, according to the present invention, has a content of isomer 15β, 16β of formula (XIX) in an amount lower than 0.5% (area %) preferably lower than 0.2% (area %) determined by HPLC analysis:

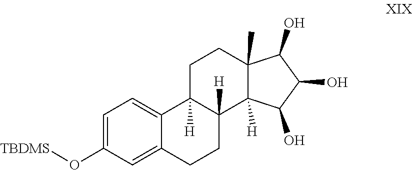

A compound of formula (IIa), where A is as defined above, in crystalline form and high diastereoisomeric purity, according to the present invention, can be subjected to a deprotection reaction of the silyl protective group of formula A to give Estetrol of formula (I) in high yield and high diastereoisomeric purity according to the processes known to the person skilled in the art.

Therefore, in another embodiment, the process of the present invention involves the further step of cleaving the protective group from the compound of formula (IIa) and preferably from the compound of formula (II) to give Estetrol of formula (I).

A further aspect of the present invention is the use of the compound of formula (IIa) and preferably of the compound of formula (II), as starting material to prepare Estetrol of formula (I).

Estetrol of formula (I), thus obtained, has a diastereoisomeric purity greater than or equal to 99.5% (area %), preferably greater than or equal to 99.9% (area %), measured by HPLC analysis.

Therefore, by using the process of the present invention, it is possible to obtain Estetrol of formula (I), in the isomerically pure form 15α, 16α which allows its use as pharmaceutical.

In particular, Estetrol of formula (I), has a content of isomer 15β, 16β of formula (Ib) in an amount lower than 0.1% (area %), preferably lower than 0.05% (area %), determined by HPLC analysis.

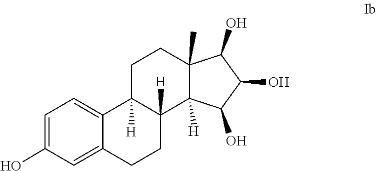

The deprotection reaction of a compound of formula (IIa), where A is as defined above, according to the present invention, is a desilylation reaction that can be performed according to processes known to the person skilled in the art, as described by Green and Wuts in “Protective Groups in Organic Synthesis”, 4th Edition (2007), Ed. John Wiley & Sons (ISBN 0-471-69754-0).

The preparation of the mixture of a compound of formula (IIa) and (IIb), to be subjected to the process of the invention, is known in the art, for example it is disclosed in WO2013/034780 previously reported in Scheme 7.

According to a particular embodiment of the present invention, the mixture of compounds of formula (IIa) and (IIb) can be prepared by cis-dihydroxylation of a compound of formula (XX), corresponding to the compound of formula (XVI) of Scheme 7, where A is as defined previously, in the presence of a catalytic quantity of K₂OsO₄·2H₂O by using trimethylamine N-oxide (TMANO) as final oxidant in a solvent, preferably methyl ethyl ketone (Scheme 9):

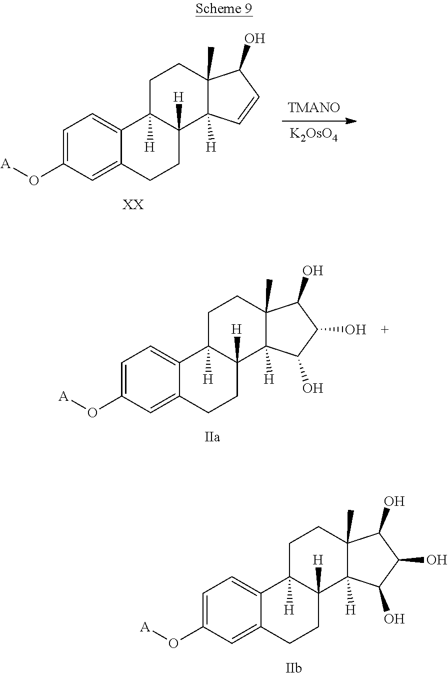

The cis dihydroxylation reaction can be performed at a temperature ranging from 35° C. to 65° C., and after aqueous washing and isolation, a mixture of a compound of formula (IIa) and its isomer of formula (IIb) is obtained, in a 90/10 ratio, determined by HPLC.

The mixture of the compound of formula (IIa) and its isomer of formula (IIb) can be isolated as a solid by known techniques, such as filtration or centrifugation. The mixture can be dried using known processes, for example oven under vacuum.

The preparation of the compound of formula (XX),

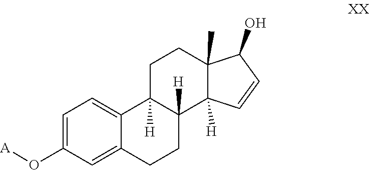

can be performed by a reduction reaction of the compound of formula (XXI) with NaBH₄ and CeCl₃ according to WO2013/034780, preferably following the procedure depicted in Scheme 10, wherein A is a silyl protecting group, as defined above:

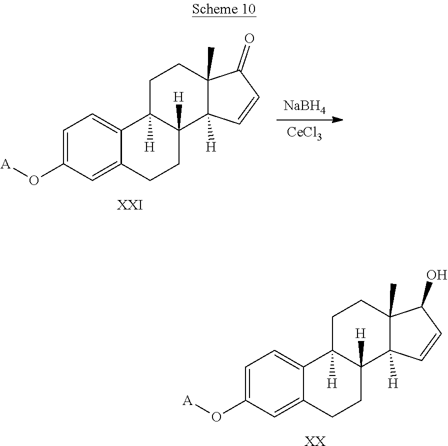

The reduction reaction can be performed in a solvent mixture containing methanol at a temperature ranging from 0° C. to 5° C., in the presence of cerium chloride heptahydrate and sodium borohydride. After common aqueous washings, the product of formula (XX) can be eventually purified by crystallization.

The compound of formula (XXI) can be prepared from Δ-15 Estrone of formula (IV) by protection reaction of the hydroxyl group in C3:

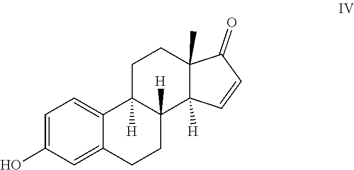

The protection reaction can be performed reacting the Δ-15 Estrone of formula (IV) with a silylating agent of formula A-X where A is as defined above and X is a leaving group selected from:
an alkylsulfonate of formula YSO₃⁻ wherein Y is a (C1-C6)alkyl group, optionally substituted by one more halogen, preferably fluoride; for example, an alkylsulfonate of formula YSO₃⁻ is methanesulfonate, or trifluoromethanesulfonate;
a halogen, preferably chloride;
in a solvent and in the presence of a base.

The reaction can be carried out under stirring at a temperature ranging from 20° C. and 25° C. and after aqueous washing of the end reaction mixture, the product of formula (XXI) can be isolated by filtration (Scheme 11):

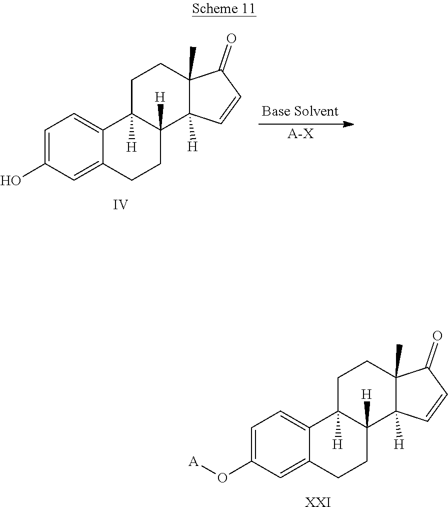

The silylating agent of formula A-X is selected from the group comprising: trimethylsilyl triflate, triethylsilyl triflate, tert-butyl dimethylsilyl triflate, triisopropylsilyl triflate, diphenylterbutylsilyl triflate, diphenylmethylsilyl triflate and dimethylphenylsilyl triflate; or can be selected from the group comprising: trimethylsilyl chloride, triethylsilyl chloride, tert-butyl dimethylsilyl chloride, triisopropylsilyl chloride, diphenyltertbutylsylyl chloride, diphenylmethylsilyl chloride, dimethylphenylsilyl chloride, preferably tert-butyldimethylsilyl chloride.

The solvent used in the protection reaction can be selected from an aprotic polar solvent, preferably dimethylformamide, a chlorinated solvent, for example dichloromethane, or an ethereal solvent.

The ethereal solvent, preferably used in the protection reaction, can be a cyclic ethereal solvent, preferably tetrahydrofuran or 1,4-dioxane or a mixture thereof, more preferably the solvent of the protection reaction is tetrahydrofuran.

The base used in the protection reaction can be an organic or inorganic base.

An organic base can be an aliphatic or aromatic amine, preferably it is selected from the group comprising 2,6-dimethylpyridine, 2,4,6-trimethylpiridine, N,N-diisopropylethylamine (DIPEA), triethylamine, methylimidazole imidazole and, preferably, 1,8-diazabiciclo [5.4.0] undec-7-ene (DBU).

An inorganic base can be selected from the group comprising a hydroxide or a carbonate of an alkaline or alkaline earth metal.

However, when the protection reaction has been carried out on the Δ-15 Estrone of formula (IV), using processes known to the person skilled in the art, using for example a silylating agent of formula A-X, for example tertbutyldimethylsilyl chloride in dimethylformamide in presence of imidazole, the yield of the protection reaction resulted to be around 50% and the HPLC profile of the product has always highlighted the presence of several steroidal impurities.

Surprisingly, the authors of the present invention have found that when the protection reaction is performed using 1,8-diazabicyclo [5.4.0] undec-7-ene (DBU) as the organic base, the yield of the protection reaction is increased up to 95%. In table 1 below the comparison between the molar yields obtained using different organic bases is reported:

TABLE 1

| Base | Molar yield |
|---|---|
| Imidazole | 45%-65% |
| Methylimidazole | 65% |
| N,N-diisopropylethylamine | <10% |
| DBU | 95% |

Therefore, another aspect of the present invention is a process for the preparation of a compound of formula (IIa) in high diastereoisomeric purity and in crystalline form, as defined above, where the mixture of the compound of formula (IIa) and its isomer of formula (IIb) is obtained starting from Δ-15 Estrone by the following steps:
a) protection of the hydroxyl group of the Δ-15 Estrone of formula (IV)

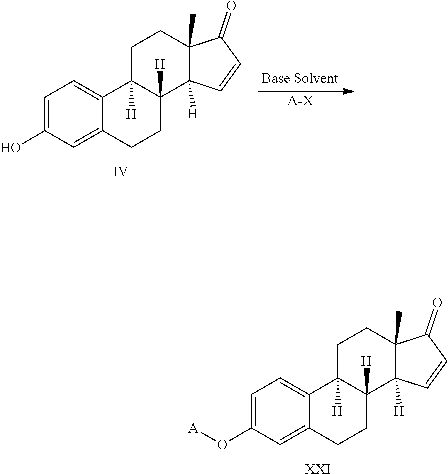

by reaction of (IV) with a silylating agent of formula A-X, where A is as defined above and X is a leaving group, in the presence of a base, to give a compound of formula (XXI);
b) reduction of the compound of formula (XXI) to give a compound of formula (XX):

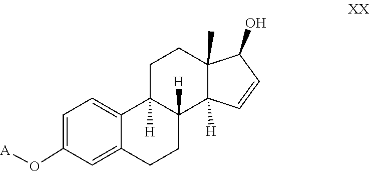

c) cis-dihydroxylation of the compound of formula (XX) to give the mixture of the compound of formula (IIa) and its isomer of formula (IIb).

In a preferred embodiment of this aspect of the invention, the base used in step a) is 1,8-diazabicyclo [5.4.0] undec-7-ene (DBU).

The preparation of Δ-15 Estrone of formula (IV) is known in the art (see for example Cantrall et al., J. Org. Chem. 1964, 29, 214-217; Johnson et al., J. Am. Chem. Soc. 1957, 79, 2005-2009; Poirier et al., Tetrahedron 1991, 47, 7751-7766; Nambara et al., Steroids 1976, 27, 111-121; Li et al.; Steroids 2010. 75, 859-869).

According to a particular embodiment, the compound of formula (IV) can be prepared starting from Estrone of formula (V), following the process depicted in Scheme 12:

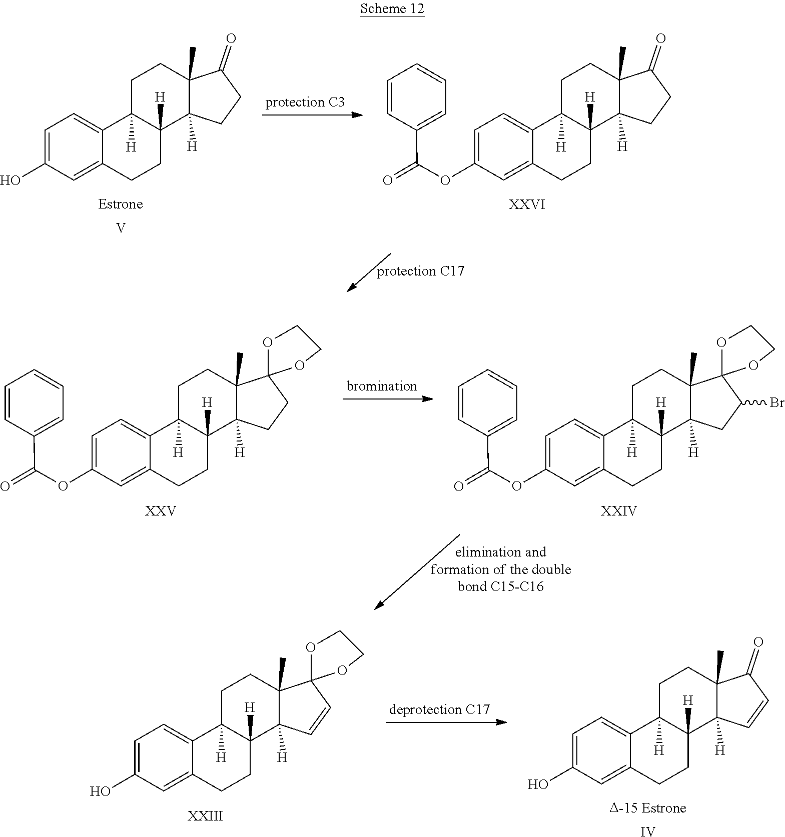

The process shown in Scheme 12 can provide the intermediate Δ-15 Estrone of formula (IV) in a high purity, typically higher than 95% (area % HPLC), and an overall yield of 65% from the starting material Estrone of formula (V).

### EXAMPLES

### Analytical Processes

The compound of formula (II), in crystalline form, herein defined as Form I, has been characterized by powder X-ray diffraction analysis (XRPD), differential scanning calorimetry (DSC), thermogravimetric analysis (TGA) and nuclear magnetic resonance (NMR).

The X-ray diffraction profile has been acquired with the X′Pert Panalytical diffractometer equipped with a copper anode (λ=1,5418 Å) and an X'Celerator detector scanning from 3° to 60° in 20 scale with one step by 0.05° for 22 sec.

The DSC thermogram has been acquired using Mettler-Toledo DSC-Q20. in open pan under flow of N₂ from 35° C. to 350° C. at 10° C./min.

The water content was determined by titration using the Karl Fisher technique and thermogravimetric analysis.

The equipment used to perform the HPLC analysis is an Agilent 1290 Infinity II equipped with an Agilent 1290 Infinity II Diode array detector. As stationary phases have been used both a C18 coreshell columns (Kinetex C18, 150 mm×4.6 mm) and a C8 columns (Kinetex C8, 150 mm×4.6 mm). The mobile phase used has been prepared with variable gradients of water/acetonitrile mixtures.

The spectroscopic analysis of Nuclear Magnetic Resonance (NMR) was carried out using an MR-400 instrument (400 MHz) with CDCl₃ as a solvent, with a delay time of 2 sec.

### Example 1—Preparation of the Compound of Formula XXVI

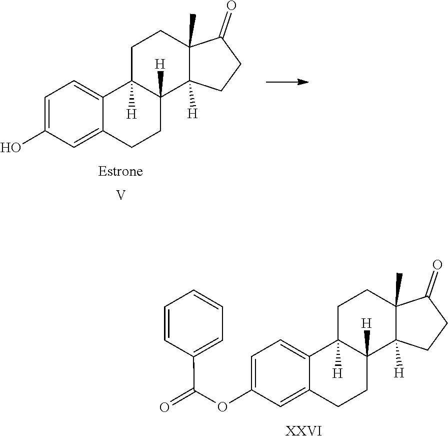

To a cooled suspension of 50.0 g of Estrone in 150 ml of THF and 50 ml of triethylamine, 24 ml of benzoyl chloride were added at a temperature below 15° C. After addition, the suspension was stirred at 20/25° C. for at least 1.5 hours. Then a solution of 200 ml of HCl 1M and 300 ml of water was added, the suspension was stirred at a temperature of 20/25° C. and the solid obtained was filtered. The solid was then washed with water and it was dried at 55° C., obtaining 71 g of compound of formula (XXVI).

Yield: quantitative. MS positive: 392 [M+NH₄]⁺; 771 [2M+Na]⁺. ¹H-NMR (400 MHZ, CDCl₃-d) δ 8.22-8.19 (m, 2H), 7.66-7.62 (m, 1H), 7.54-7.49 (m, 2H), 7.35 (dd, 1H, J=8.8 Hz and 1.2 Hz), 7.07-6.96 (m, 2H), 2.98-2.94 (m, 2H), 2.56-2.42 (m, 2H), 2.36-2.29 (m, 1H), 2.21-1.97 (m, 4H), 1.75-1.43 (m, 6H), 0.94 (s, 3H).

### Example 2—Preparation of the Compound of Formula XXV

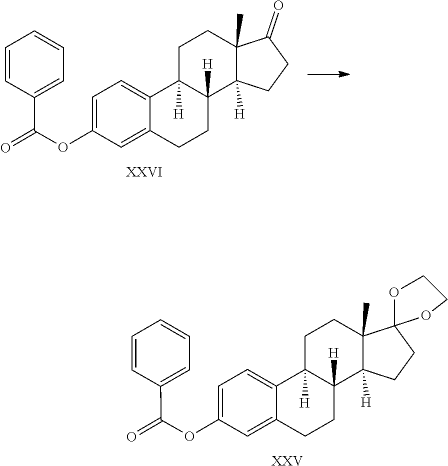

71 g of compound of formula (XXVI) were suspended in 142 ml of ethylene glycol and 35.5 ml of triethylortoformate, and then 1.42 g of p-toluenesulfonic acid were added. The suspension was stirred at 40/45° C. for at least 4 hours then the reaction mixture was cooled to a temperature of 20/25° C. and then 2.5 ml of triethylamine were added. The suspension was stirred for 10-15 minutes and then 710 ml of water were added; the suspension was stirred for two hours at 20-25° C. and filtered. The solid was washed with water and it was dried at 55° C., obtaining 79 g of compound of formula (XXV).

Yield: quantitative. 1H-NMR NMR (400 MHZ, CDCl₃-d) δ 8.215 (dd, 2H, J=8.4 Hz and 1.2 Hz), 7.66-7.61 (m, 1H), 7.54-7.49 (m, 2H), 7.35 (dd, 1H, J=8.6 Hz and 1.0 Hz), 6.99-6.94 (m, 2H), 4.00-2.89 (m, 4H), 2.97-2.89 (m, 2H), 2.43-2.28 (m, 2H), 2.10-1.76 (m, 5H), 1.71-1.32 (m, 6H), 0.92 (s, 3H).

### Example 3—Preparation of the Compound of Formula XXIV

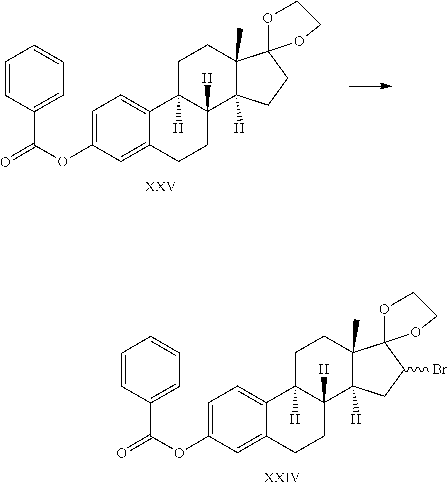

73 g of compound of formula (XXV) were suspended in 237 ml of THF and 15.8 ml of ethylene glycol, then a solution of 109 ml of THF and 79.6 g of phenyl trimethylammonium tribromide was added at a temperature of 20/25° C. The suspension was stirred at 20/25° C. for at least 1 hour and then a solution of 42 g of sodium thiosulfate pentahydrate and 7.3 g of sodium bicarbonate in water was added to the reaction mixture. The suspension was stirred at 20/25° C. for at least 1 hour and filtered. The solid was washed with water, then purified by crystallization from acetone. The solid was then dried at 55° C., obtaining 84 g of compound of formula (XXIV).

Yield: 96.8%. ¹H-NMR (400 MHZ, CDCl₃-d) δ 8.22-8.19 (m, 2H), 7.66-7.62 (m, 1H), 7.53-7.49 (m, 2H), 7.33 (d, 1H, J=8.4 Hz), 6.99 (dd, 1H, J=8.4 Hz and 2.4 Hz), 6.95-6.93 (m, 1H), 4.57 (dd, 1H, J=10.6 Hz and 4.0 Hz), 4.32-4.26 (m, 1H), 4.26-4.14 (m, 1H), 4.04-3.94 (m, 2H), 2.91-2.88 (m, 2H), 2.39-2.09 (m, 4H), 2.03-1.80 (m, 3H), 1.62-1.36 (m, 4H), 0.94 (s, 3H).

### Example 4—Preparation of the Compound of Formula XXIII

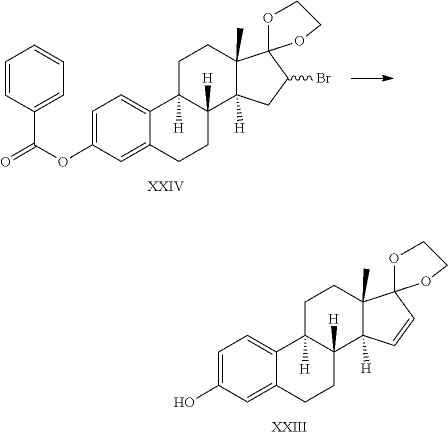

65 g of compound of formula (XXIV) were suspended in 323 ml of DMSO, then 58.2 g of potassium tert-butoxide were added. The suspension was stirred at 50/55° C. for at least 3 hour and then the reaction mixture was cooled to a temperature of 20/25° C., then water was added, and the pH was corrected with acetic acid to neutrality. The final suspension was stirred at 20/25° C. for 1 hour and filtered off. The solid was washed with water, suspended again in water, stirred for 1 hour and filtered. The solid was then dried at a temperature of 55° C., obtaining 36 g of compound of formula (XXIII).

Yield: 88.6%. MS positive: 313 [M+H]⁺; 624 [2M+H]⁺. ¹H-NMR (400 MHZ, DMSO-d6) δ 8.99 (s, 1H), 7.03 (dd, 1H, J=8.6 Hz and 1.0 Hz), 6.51 (dd, 1H, J=8.4 Hz and 2.8 Hz), 6.45 (d, 1H, J=2.6 Hz), 6.24 (dd, 1H, J=6.0 Hz and 1.6 Hz), 5.71 (dd, 1H, J=6.0 Hz and 3.2 Hz), 3.94-3.72 (m, 4H), 2.82-2.62 (m, 2H), 2.33-2.27 (m, 2H), 2.18-2.12 (m, 1H), 2.05-1.99 (m, 1H), 1.84 (dt, 1H, J=12.4 Hz and 4.4 Hz), 1.56-1.28 (m, 4H), 0.85 (s, 3H).

### Example 5—Preparation of the Compound of Formula IV

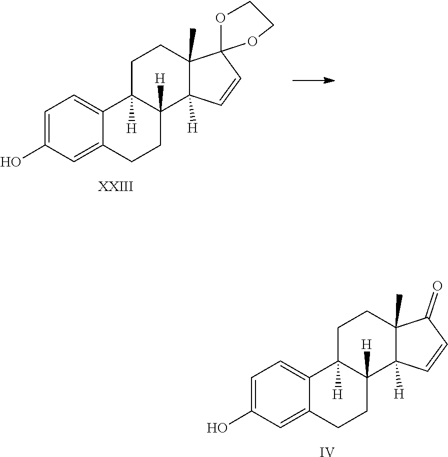

36 g of compound of formula (XXIII) were suspended in 250 ml of acetone and 36 ml of a solution of sodium bisulfate. The suspension was stirred at a temperature of 20/25° C. for at least 2.5 hours and then a saturated solution of 30 ml of NaCO₃ and 450 ml of water were added. The mixture was stirred for two hours at a temperature of 20/25° C. and then the solid was filtered. The solid was washed with water and dried at 55° C., obtaining 28 g of the Δ-15 Estrone of formula (IV).

Yield: 90%. MS positive: 269 [M+H]⁺; 537 [2M+H]⁺; 559 [2M+Na]⁺. ¹H-NMR (400 MHz, DMSO-d6) δ 9.02 (s, 1H), 7.83 (dd, 1H, J=6.0 Hz and 2.0 Hz), 7.04 (dd, 1H, J=8.8 Hz and 1.6 Hz), 6.53 (dd, 1H, J=8.4 Hz and 2.6 Hz), 6.47 (d, 1H, J=2.6 Hz), 6.05 (dd, 1H, J=6.0 Hz and 3.2 Hz), 2.86-2.74 (m, 2H), 2.52-2.48 (m, 1H), 2.37-2.31 (m, 1H), 2.27-2.14 (m, 2H), 1.81-1.76 (m, 1H), 1.72-1.61 (m, 2H), 1.57-1.36 (m, 2H), 0.98 (s, 3H).

### Example 6-Preparation of the Compound of Formula (XXI), where a is tert-butyldimethylsilyl

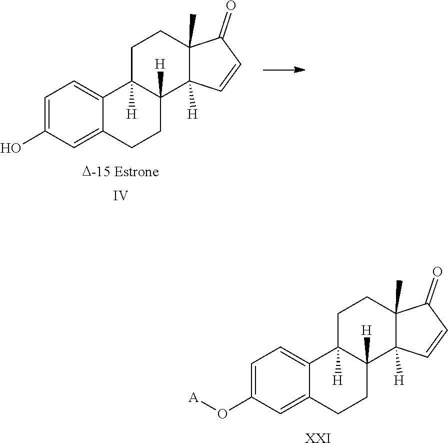

200 g of Δ-15 Estrone of formula (IV) and 124.0 g of TBDMSCI were suspended at a temperature of 0/5° C. in 800 ml of THE, then was added a solution of 126 g of DBU diluted with 200 ml of THF keeping the temperature below 10° C. The suspension was stirred at a temperature of 20/25° C. for at least 1 hour and then 2000 ml of water were added to the reaction mixture and the obtained suspension was stirred for two hours at a temperature of 20/25° C. and filtered. The solid was washed with water and dried at 55° C., obtaining 280 g of compound of formula (XXI).

Yield: 98%. ¹H-NMR (400 MHz, DMSO-d6+CDCl₃-d) δ 7.73-7.71 (m, 1H), 7.11-7.06 (m, 1H), 6.55 (dd, 1H, J=8.4 Hz and 1.2 Hz), 6.51-6.50 (m, 1H), 6.00 (gg, 1H, J=6.0 Hz and 3.2 Hz), 2.90-2.78 (m, 2H), 2.47-2.06 (m, 4H), 1.86-1.33 (m, 5H), 1.01 (s, 3H), 0.93 (s, 9H), 0.13 (s, 6H).

### Example 7.—Preparation of the Compound of Formula (XX), where a is tert-butyldimethylsilyl

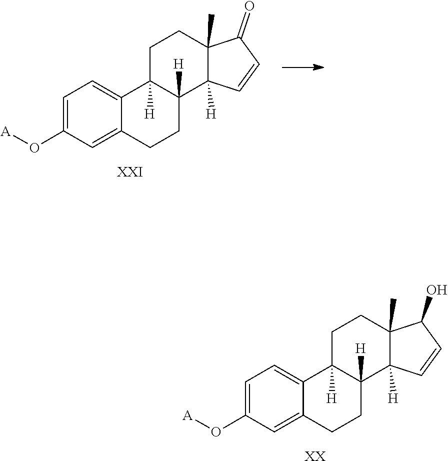

280 g of compound of formula (XXI) were suspended at 0/5° C. in a mixture of 950 ml of THF and 950 ml of methanol, then 84 g of cerium chloride heptahydrate and 9.4 g of sodium borohydride were added. The suspension was stirred at a temperature of 0/5° C. for at least 15 minutes and then the pH was corrected with acetic acid to neutrality, then 2800 ml of water were added at a temperature of 20/25° C. The final suspension was stirred at a temperature of 20/25° C. for at least 1 hour and filtered off. The solid was washed with water and dried at 55° C., obtaining 312 g of crude compound of formula (XX) than purified by crystallization from an acetone/methanol mixture. The solid was then dried at 55° C., to afford a final dry cake: 246.0 g of the compound of formula (XX).

Yield: 87.4%. ¹H-NMR (400 MHZ, CDCl₃-d) δ 7.14-7.12 (m, 1H), 6.64 (dd, 1H, J=8.4 Hz and 2.4 Hz), 6.58 (d, 1H, J=2.4 Hz), 6.05-6.03 (m, 1H), 5.75-5.72 (m, 1H), 4.42-4.40 (m, 1H), 2.94-2.81 (m, 2H), 2.37-2.23 (m, 2H), 2.11-2.00 (m, 3H), 1.72-1.45 (m, 5H), 1.00 (s, 9H), 0.88 (s, 3H), 0.21 (s, 6H).

### Example 8—Preparation of the Mixture Containing a Compound of Formula (IIa) and its Isomer of Formula (IIb) in the Ratio 90/10. Where a is Tert-Butyldimethylsilyl

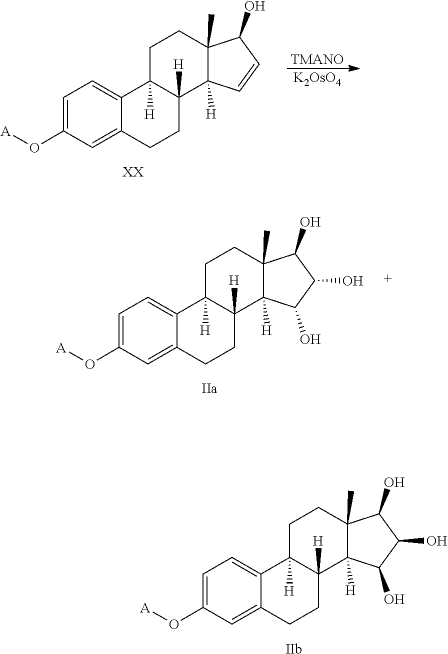

50 g of compound of formula (XX) were suspended at a temperature of 20/25° C. in 200 ml of methyl ethyl ketone (MEK) and 5 ml of water, then 13.5 g of TMANO dihydrate and 0.1 g of Potassium osmate dihydrate were added. The reaction mixture was stirred 5 hours at a temperature of 50/55° C., then the reaction mixture was cooled to a temperature of 20/25° C. and then a solution of 17 g of sodium metabisulfite was added. The suspension was stirred for 10-15 minutes and then 200 ml of ethyl acetate were added, the two phases were separated and the aqueous phase was extracted with ethyl acetate while the resulting organic phase was concentrated under vacuum to obtain 50 g of a solid mixture of a compound of formula (IIa) and its isomer of formula (IIb). The ratio between the α-15,16-diol and β-15,16-diol is 90/10.

### Example 9-Preparation of the Compound of Formula (IIa) where a is Tertbutyldimethylsilyl

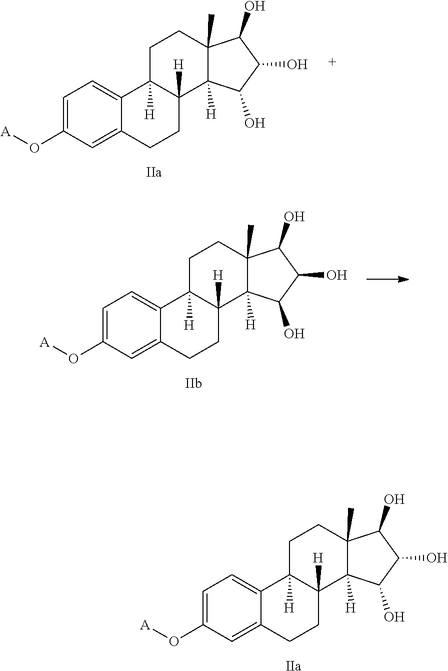

1.0 g of a mixture of a compound α-15,16-diol of formula (IIa) and β-15,16-diol of formula (IIb) in a 90/10 ratio was dissolved at a temperature of 50/55° C. in 3 ml of THF and the solution was then cooled to a temperature of 20/25° C., then 18 ml of diisopropyl ether were added. The obtained suspension was stirred for 1 hour at a temperature 20/25° C., filtered off, washed with diisopropyl ether and dried at a temperature of 55° C., obtaining 0.37 g of compound of formula (IIa). The ratio between the α-15,16-diol of formula (IIa) and the β-15,16-diol of formula (IIb) is 99.8/0.2.

Yield: 37%. MS positive: 419 [M+H]⁺; 436 [M+NH₄]⁺. ¹H-NMR (400 MHZ, DMSO-d6) δ 7.109 (d, 1H, J=8.4 Hz), 6.568 (dd, 1H, J=8.4 and 2.4 Hz), 6.510 (d, 1H, J=2.8 Hz), 4.727 (d, 1H, J=4.8 Hz), 4.508 (d, 1H, J=5.6 Hz), 4.155 (d, 1H, J=5.6 Hz), 3.756-3.646 (m, 2H), 3.265 (t, 1H, J=5.6 Hz), 2.770-2.730 (m, 2H), 2.257-2.091 (m, 3H), 1.796-1.750 (m, 1H), 1.544-1.455 (m, 1H), 1.409-1.146 (m, 3H), 1.099-1.037 (m, 1H), 0.945 (m, 9H), 0.683 (s, 3H), 0.159 (m, 6H).

### Example 10-Preparation of the Compound of Formula (IIa) where a is Tertbutyldimethylsilyl

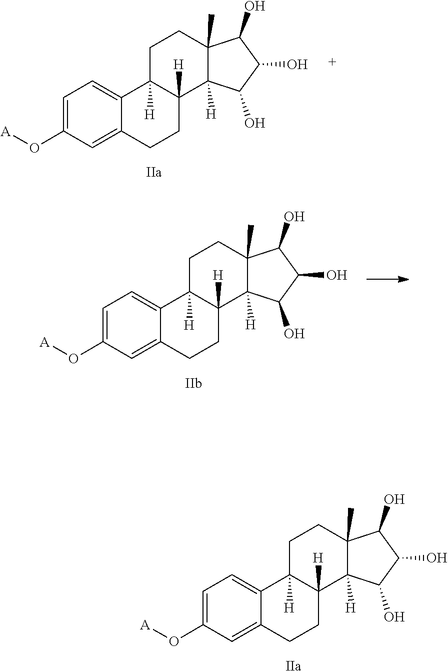

50 g of a mixture of a compound α-15,16-diol of formula (IIa) and β-15,16-diol of formula (IIb) in a 90/10 ratio were dissolved at a temperature of 50/55° C. in 75 ml of THF, then the solution was cooled to a temperature of 20/25° C., then 450 ml of diisopropyl ether were added. The obtained suspension was concentrated under vacuum, stirred for 1 hour at a temperature of 20/25° C. and filtered off. The wet cake of the product of formula (IIa) was washed with diisopropyl ether and dried at a temperature of 55° C., obtaining 35.6 g of a compound of formula (IIa) which can be recrystallized under the same conditions, obtaining 32.6 g of a compound of formula (IIa) where the ratio between the α-15, 16-diol and β-15, 16-diol is 99.8/0.2.

Yield: 65%. MS positive: 419 [M+H]⁺; 436 [M+NH4]⁺. ¹H-NMR (400 MHZ, DMSO-d6) δ 7.11 (d, 1H, J=8.4 Hz), 6.57 (dd, 1H, J=8.4 and 2.4 Hz), 6.51 (d, 1H, J=2.8 Hz), 4.73 (d, 1H, J=4.8 Hz), 4.51 (d, 1H, J=5.6 Hz), 4.15 (d, 1H, J=5.6 Hz), 3.76-3.65 (m, 2H), 3.26 (t, 1H, J=5.6 Hz), 2.77-2.73 (m, 2H), 2.26-2.09 (m, 3H), 1.80-1.75 (m, 1H), 1.54-1.45 (m, 1H), 1.41-1.15 (m, 3H), 1.01-1.04 (m, 1H), 0.94 (m, 9H), 0.68 (s, 3H), 0.16 (m, 6H).

### Example 11—Preparation of Estetrol

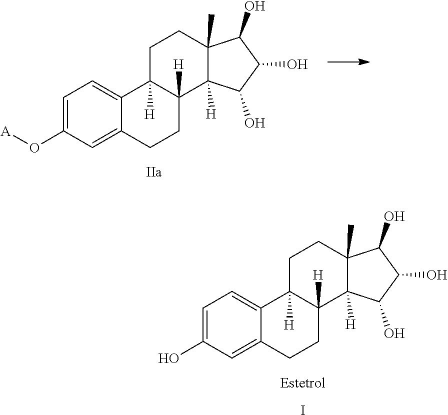

10.0 g of purified compound of formula (IIa), where A is tert-butyldimethylsilyl, were suspended in 60 ml of methanol, then 10 ml of 30% (p/p) aqueous sodium hydroxide solution were added. The suspension was stirred at a temperature of 20/25° C. for at least 1 hour and then a solution of 10 ml of acetic acid 1M and 180 ml of water were added. The final suspension was stirred for two hours at a temperature of 20/25° C. and then the solid was filtered off, washed with water and dried at a temperature of 55° C., obtaining 7.3 g of Estetrol of formula (I).

Yield: 99.8%. MS positive: 305 [M+H]⁺; 322 [M+NH4]⁺; 327 [M+Na]⁺; 344 [M+K]⁺; 631 [2M+Na]⁺. ¹H-NMR (400 MHZ, DMSO-46) δ 8.86 (brs, 1H), 7.03 (d, 1H, J=8.4), 6.50 (dd, 1H, J=8.4 Hz and 2.8 Hz), 6.44 (brs, 1H), 4.73 (re, 1H, J=4.8 Hz), 4.50 (brs, 1H), 4.15 (brs, 1H), 3.75-3.65 (m, 2H), 3.26 (brs, 1H), 2.78-2.65 (m, 2H), 2.24-2.18 (m, 2H), 2.20-2.05 (m, 1H), 1.78-1.74 (m, 1H), 1.51-1.43 (m, 1H), 1.38-1.13 (m, 3H), 1.08-1.03 (m, 1H), 0.68 (s, 3H).

1. A process for the preparation of a compound of formula (IIa) in high diastereoisomeric purity and in crystalline form 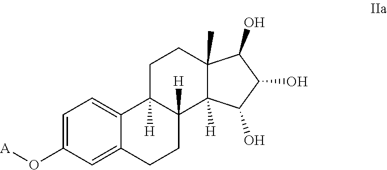
comprising
crystallizing a mixture of the compound of formula (IIa) and its isomer of formula (IIb) 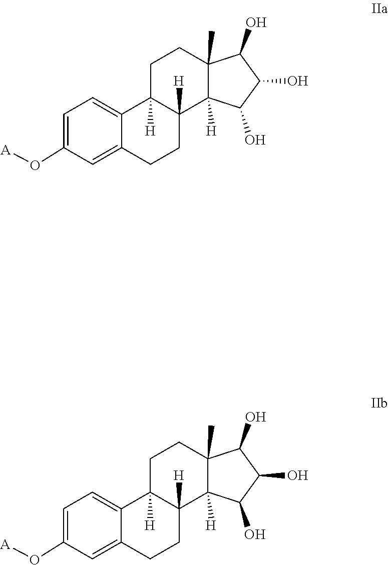
from an ether solvent comprising at least one linear or branched di(C1-C5)alkyl ether and at least one cyclic (C4-C6)cycloaliphatic ether,
wherein A is a protective group of formula —Si(R)3, wherein R is selected independently from the group comprising (C1-C4)alkyl, (C6-C10) aryl, (C1-C4)alkyl-(C6-C10) aryl and (C6-C10) aryl-(C1-C4)alkyl.

2. The process according to claim 1, wherein A is selected from the group consisting of trimethylsilyl, triethylsilyl, tert-butyldimethylsilyl, tri-isopropylsilyl, diphenyltertbutylsilyl, diphenylmethylsilyl and phenyldimethylsilyl.

3. The process according to claim 1, wherein the ether solvent consists of a mixture of a (C4-C6)cycloaliphatic ether and a linear or branched di(C1-C5)alkyl ether.

4. The process according to claim 3, wherein the ratio between the (C4-C6)cycloaliphatic ether and the linear or branched di(C1-C5)alkyl ether is from 1:1 to 1:10 expressed as v/v.

5. The process according to claim 3, wherein the (C4-C6)cycloaliphatic ether is tetrahydrofuran and the linear or branched di(C1-C5)alkyl ether is diisopropyl ether.

6. The process according to claim 1, wherein the mixture of compounds (IIa) and (IIb) is obtained through the following steps:
a) protecting hydroxyl group of Δ-15 estrone of formula (IV) 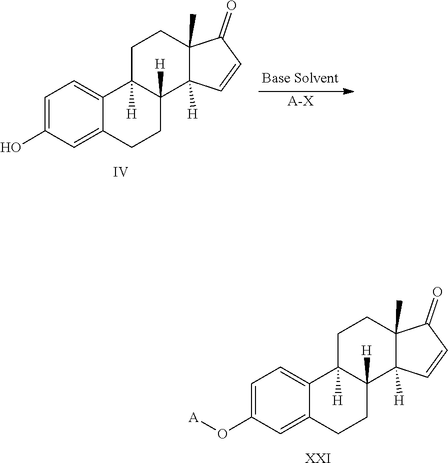
by reacting said compound of formula (IV) with a silylating agent of formula A-X, wherein A is as defined above and X is a leaving group, in the presence of a base, to obtain a compound of formula (XXI);
b) reducing the compound of formula (XXI) to obtain a compound of formula (XX) 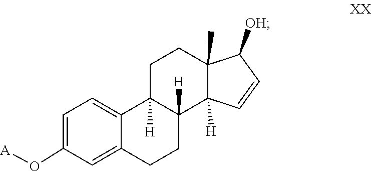
c) cis-dihydroxylating the compound of formula (XX) to obtain the mixture of the compound of formula (IIa) and its isomer of formula (IIb).

7. The process according to claim 6, wherein the base used in step a) is 1,8-diazabicyclo [5,4.0] undec-7-ene (DBU).

8. The process according to claim 1, further comprising removing the protective group A from the compound of formula (IIa), to obtain Estetrol of formula (I)

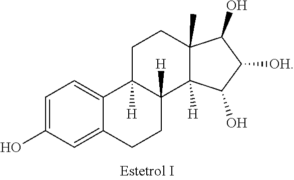

9. Compound of formula (IIa) 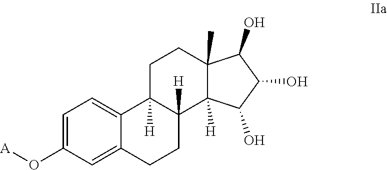
wherein A is a protective group of formula —Si(R)3, wherein R is selected independently from the group comprising (C1-C4)alkyl, (C6-C10)aryl, (C1-C4)alkyl-(C6-C10) aryl and (C6-C10) aryl-(C1-C4)alkyl, wherein as defined in the preceding claims, characterized in that
said compound of formula IIA
(i) is in crystalline form,
(ii) has a diastereomeric purity as determined by HPLC analysis of at least 99.5% (area %) and
(iii) contains less than 0.5% (area %) of compound of formula (IIb)

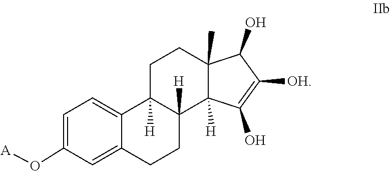

10. The compound according to claim 9, having formula (II) 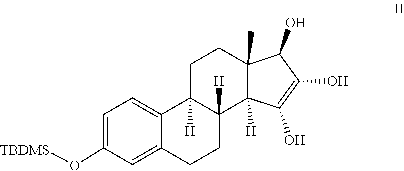
wherein TBDMS represents tert-butyldimethylsilyl group, wherein the crystalline form has an XRPD profile comprising the most intense peaks at 20 angles of 6.8±0.2°, 10.2±0.2°, 13.6±0.2°, 14.6±0.2° and 15.8±0.2°, measured at a wavelength of 1.5418 Å.

11. A process for the preparation of Estetrol of formula (I) 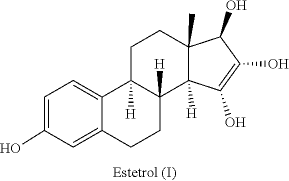
with the compound of formula (IIa) as defined in claim 9 as starting material 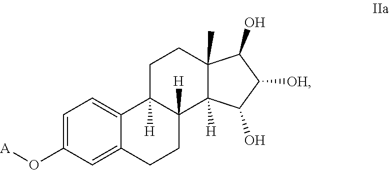
said method comprising
crystallizing a mixture of the compound of formula (IIa) and its isomer of formula (IIb) 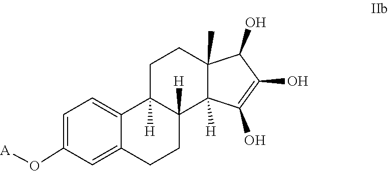
from an ether solvent comprising at least one linear or branched di(C1-C5)alkyl ether and at least one cyclic (C4-C6)cycloaliphatic ether,
wherein A is a protective group of formula —Si(R)3, wherein R is selected independently from the group comprising (C1-C4)alkyl, (C6-C10)aryl, (C1-C4)alkyl-(C6-C10)aryl and (C6-C10)aryl-(C1-C4)alkyl; and
further removing the protecting group A from the compound of formula (IIa) to obtain Estetrol of formula (I).

12. The method according to claim 11, wherein in compound (IIa) the group A is tert-butyldimethylsilyl and the crystalline form has an XRPD profile comprising the most intense peaks at 2θ angles of 6.8±0.2°, 10.2±0.2°, 13.6±0.2°, 14.6±0.2° and 15.8±0.2°, measured at a wavelength of 1.5418 Å.

13. The process according to claim 2, wherein A is tert-butyldimethylsilyl.

14. The process according to claim 4, wherein the ratio between the (C4-C6)cycloaliphatic ether and the linear or branched di(C1-C5)alkyl ether is from 1:3 to 1:7.
